(19) United States
(12) Patent Application Publication
(10) Pub. No.: US 2012/0157975 A1
(43) Pub. Date: Jun. 21, 2012

(54) Catheters

(76) Inventor: Roger Charles Leslie Feneley, Clifton (GB)

(21) Appl. No.: 13/391,866

(22) PCT Filed: Aug. 11, 2010
(86) PCT No.: PCT/GB10/01523

§ 371 Date: Feb. 23, 2012

(30) Foreign Application Priority Data
Aug. 24, 2009 (GB) 0914766.1

(51) Int. Cl. A61M 27/00

(52) U.S. Cl. 604/544

(57) ABSTRACT

A suprapubic urethral catheter has a hollow tube with a suprapubic section (1), a bladder section (2) and a urethral section (3). An abdominal stop (5) is slidably mounted on the suprapubic section (1), and the bladder section (2) has drainage eyeholes (4) which enable urine from the bladder to drain into the catheter. A non-return cuff (6) is mounted on the bladder section between the drainage eyeholes (4) and the abdominal stop, the non-return cuff (6) having a projecting portion projecting outwards from the tube, being deformable towards the tube and resisting deformation away from the tube. The cuff (6) allows the catheter to be inserted into a suprapubic track in the abdominal wall of a user, but once the cuff has entered the bladder, the projecting portion prevents retrograde movement of the catheter out of the suprapubic track.

### BACKGROUND OF THE INVENTION

1. Field of Invention

The present invention relates to catheters for drainage of the urinary bladder. In particular, the invention relates to suprapubic urethral catheters and catheter systems.

2. Summary of the Prior Art

Urinary catheterisation introduces major medical, social and economic issues. The catheter is a medical device used to drain urine from the bladder but it also provides a conduit for bacteria to enter the body. One out of 4 hospitalized patients receives an indwelling catheter [1]. Catheter-associated urinary tract infections present the most common nosocomial infections, developing at an estimated rate of 5%. per day and increasing the risk of bacteraemia and mortality [2]. Financial estimates in 2000 suggested that a symptomatic urinary tract infection raises the cost of care by around £420 and bacteraemia to at least £1750 by lengthening the stay in hospital and antibiotic therapy [3]. In developing countries the rates of healthcare-associated infections are 3 to 5 times higher than international standards [4]

The self-retaining urinary catheter introduced by Dr Foley in 1937 has been the standard product in routine use for over 70 years [5]. The catheter is retained in the bladder by means of an inflatable balloon but this prevents the bladder from emptying completely, leaving residual infected urine. Long-term urinary catheterisation (LTC) provides a well-established method of management, draining urine from the bladder into a urine collection bag. LTC carries a high risk of complications; catheter-associated complications occur in over 70% of patients, the majority resulting from the rapid bacterial invasion of the catheter [6]. Urinary tract infection can lead to life threatening septicaemia but the common problems of encrustation and blockage of the catheter, urinary leakages and discomfort from the catheter have an immense impact not only on an individual's quality of life but on the costs to healthcare services [7]. The prevalence of LTC increases with age and although considered a last resort, many thousands of people rely on this method of urinary collection because no acceptable alternative is available [8].

A Foley-type catheter is usually positioned in a patient by passing it up through the urethra. Such a catheter is known as a urethral catheter. However, Foley-type catheters may alternatively be inserted into the bladder through the abdominal wall just above the pubic bone i.e. along a suprapubic track. The latter is known as a suprapubic catheter.

Suprapubic urethral catheters are also known in the art. For example, see GB2343847A (also by the present inventor). A suprapubic urethral catheter is positioned in a patient via a suprapubic track and enables either suprapubic or urethral drainage of a urinary bladder. A suprapubic urethral catheter generally consists of a tube having, sequentially, a suprapubic section that will be located suprapubically in a patient, a bladder section that will be located within the bladder of the patient, and a urethral section that will be located within the urethra of the patient.

### SUMMARY OF THE INVENTION

The present invention aims to overcome at least some of the aforementioned deficiencies in conventional self-retaining Foley-type catheters.

The present invention has been developed particularly for those patients who are experiencing complications with the conventional self-retaining Foley-type catheter and urine collection bag. Recurrent blocking of the catheter and bypassing of urine around the catheter are common problems associated with long-term urinary catheterisation. In addition the present invention provides a versatile method of urinary drainage and collection and may be indicated in patients presenting specific needs for urine collection. A system that mimics the cyclical filling and emptying of the bladder has been advocated to reduce the risk of urinary infections and the incidence of catheter blockages [9]; the present invention has been designed to provide this.

In a first aspect, the present invention provides a new type of self-retaining mechanism for a suprapubic urethral catheter having a novel arrangement for retaining the bladder section of the suprapubic urethral catheter in the bladder.

At its most general, the first aspect of the present invention proposes that a non-return cuff is mounted on a section of a catheter which is to be inserted into a bladder. The non-return cuff is deformable towards the catheter so that it may be inserted into the bladder without significant resistance through the suprapubic tract in the abdominal and bladder walls (e.g. when the catheter is inserted from a suprapubic direction). The projecting cuff then may prevent or resist the catheter being withdrawn from the bladder in a suprapubic direction. The bladder section of the catheter will then normally have holes (e.g. drainage eyes) for ingress of urine to the catheter duct (which duct may be referred to as a drainagelumen).

Thus, the first aspect of the present invention may provide a suprapubic urethral catheter comprising a hollow tube having sequentially a suprapubic section, a bladder section and a urethral section; wherein, there is an abdominal stop attached to the suprapubic section; and wherein, the bladder section comprises one or more drainage holes in the wall of the tube and there is a non-return cuff attached to the bladder section between the one or more drainage holes and the abdominal stop; and wherein, the non-return cuff has a projecting portion projecting outwardly from the tube which is deformable towards the tube and which resists deformation away from the tube.

The projecting portion projecting outwardly from the tube of the non-return cuff is deformable towards the tube, e.g. on insertion of the cuff into a suprapubic track in the abdominal wall of a user and into the bladder. However, once the cuff has entered the bladder, the projection portion springs outwardly once again and resists deformation away from the tube. Therefore if the catheter is urged in the retrograde direction, i.e. back out of the suprapubic track along which it was inserted, the cuff will not pass back through the entry aperture to the bladder and the cuff does not move relative to the bladder section, therefore the cuff prevents the bladder section from extruding retrogradely out of the bladder.

In other words, the non-return cuff is arranged on the bladder section of the catheter so that it is flexible enough to be inserted into the bladder via a suprapubic track in the abdominal wall of a user but the non-return cuff is stiff enough to prevent the bladder section from extruding retrogradely, i.e. backwards, out of the bladder along the suprapubic track.

The first aspect of the present invention enables a catheter to be retained in the bladder without a balloon, enabling the bladder to fill and empty at low pressure and thus matching as closely as possible the normal physiological and mechanical characteristics of the normal bladder. The regular cycle of filling and emptying provides the chief defense mechanism against the onset of urinary tract infection.

The non-return cuff may be made from any material having suitable properties (e.g. medical grade silicone), and arranged in any appropriate structural configuration to provide the necessary flexibility and stiffness. The non-return cuff may be fixed to the bladder section of a catheter using a suitable adhesive (e.g. a medical grade silicone glue), or may be integral with the bladder section.

Various shapes and structures for the non-return cuff are envisaged. For example, the projecting portion of the non-return cuff may comprise a wing, leg or arm, inclined away from the tube, wherein the end of the wing, leg or arm, furthest from the tube is nearest the abdominal stop. Alternatively, the projection portion may be of a frusto-conical shape.

Preferably, the projecting portion of the cuff comprises two inclined wings. More preferably the non-return cuff comprises a channel for receiving the tube of the catheter, and the two inclined wings are positioned on opposing sides of the channel. The two inclined wings may be joined by a resiliently deformable element and if so, the wings are biased apart from each other by the deformable element.

At the end of the wings furthest from the abdominal stop, the non-return cuff may have a sleeve formed around the channel and attached to the tube. In this embodiment, at the end of the sleeve furthest from the abdominal stop, the walls of the sleeve may taper towards the tube. This tapering is for easing insertion of the cuff into the suprapubic track and into the bladder.

Each wing may be shaped as a distorted elongated polygon, (preferably a hexagon), wherein the polygon is elongated along the length of the wing, and the widest part of the polygon is found towards the end of the wing being furthest from the abdominal stop. This shape balances the need for strength to resist deformation away from the tube with the need for ease of insertion.

Preferably the catheter comprises an irrigation hole in the wall of the tube positioned in the bladder section within the projecting portion of the non-return cuff. This enables any accumulated debris to be washed out.

Usually the catheter comprises a plurality of drainage holes or eyeholes located along the bladder section. Preferably at least one drainage hole, most preferably the drainage hole nearest the urethral section of the catheter (that is, furthest downstream) is retainable in a position at the lowest point in the bladder. This enables the bladder to be drained completely of urine, or allows only a minimal volume of residual urine to remain in the bladder on draining, and therefore reduces the risk of infection. One or more drainage holes may be positioned in the bladder section within the region of the non-return cuff to ensure drainage of residual urine from that cuff.

The abdominal stop, attached to the suprapubic section of the catheter, may be in the form of a flange. It may have a channel for receiving and gripping an external part of the suprapubic section of the catheter. Preferably the position of the abdominal stop along the catheter is adjustable, more preferably it is slidably adjustable. The channel through the flange preferably has an oval hole which can be released by simultaneous pressure on the both lateral ends of the flange to allow adjustment of its position on the catheter.

Once the catheter is in situ in a patient, the abdominal stop remains external to the body. The position of the abdominal stop can then be adjusted and held against the abdominal wall to prevent the catheter from moving forwards out of the body via the urethra.

Use of the abdominal stop together with the non-return cuff means that in situ, both forward and retrograde movement of the catheter is prevented. Therefore the catheter is securely held in position. For example, this ensures that the drainage holes in the bladder section remain in an optimum position for maximum drainage of the bladder, and overall comfort for the patient may be improved.

There may be a marker at the junction between the bladder section and the urethral section of the catheter. Such a marker is identifiable when in situ in a patient using e.g. radiological or ultrasound means. This helps to ensure the bladder section is implanted in the correct position in the patient or user.

In preferred embodiments, the catheter consists of a flexible silicone tube, 65-67 cm long and 14 FG/Ch size.

The suprapubic section is preferably 20 cm in length. The upper end of the suprapubic section can be cut to a suitable length for the user and may be fitted to an abdominal urine collection pouch or a drainage funnel or connector. Any drainage funnel or connector at the suprapubic end of the catheter can be used to irrigate or drain the bladder.

The bladder section is provided in various lengths to fit different sizes of bladder. The preferred lengths for the bladder section are 5 cm and 7 cm.

The urethral section is preferably 40 cm in length but can be cut to a suitable length to fit into a drainage funnel or connector which can be fitted e.g. with a standard catheter valve. The tubing of this section of the catheter may be thinner than the other sections of the catheter. Thus the catheter can be used for draining urine from the bladder assisted by gravity by opening a manually operable catheter valve fitted to the drainage funnel or connector at the urethral end of the catheter.

The first aspect of the present invention also provides a kit of parts for assembling a self-retaining suprapubic urethral catheter, the kit comprising:

a suprapubic urethral catheter comprising a hollow tube having sequentially a suprapubic section, a bladder section and a urethral section, wherein the bladder section comprises one or more drainage holes in the wall of the tube;
an abdominal stop attachable to the suprapubic section;
a non-return cuff attachable to the bladder section between the one or more drainage holes and the abdominal stop, wherein the non-return cuff has a projecting portion projecting outwardly from the tube which is deformable towards the tube and which resists deformation away from the tube.

In a second aspect, the present invention is concerned with providing a urine collection system having a low pressure reservoir which enables the functional capacity of the bladder to be increased. In other words an upstream overflow tank is provided to relieve pressure in the system.

Thus, according to a second aspect of the present invention, the overflow tank is in the form of a reservoir connected to a suprapubic section of a catheter which is to extend into the bladder of a patient. Thus, the second aspect of the invention may provide a urine collection system comprising:

a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, providing a flowpath from the bladder section to the container, and thus providing a low pressure reservoir.

If pressure in the bladder rises above a threshold pressure, urine flows upstream and enters the urine collection container and then returns to the bladder when the pressure in the bladder falls below the threshold. For example, if pressure in the bladder rises as a result of a contraction of the bladder (detrusor) muscle, urine enters the urine collection container and then returns to the bladder as the bladder muscle relaxes.

Many patients requiring long-term catheterization suffer from a neurological disease or injury which accounts for their loss of bladder control. Under these conditions, the bladder can become overactive with inappropriate uncontrolled contractions both of the bladder and the sphincter closing the urethral passage during filling. This is termed detrusor sphincter dysynergia. These contractions cause the pressure to rise in the bladder which in turn creates back-pressure on the kidney(s) with the risk of serious damage to their function. The objective of the upstream urine collection container is to obviate the pressure rise by providing a low pressure urinary reservoir.

Preferably, the urine collection container is attachable to the abdomen of a patient, more preferably via a waistband. Preferably the urine collection container is a pouch, most preferably it is an abdominal urine collection pouch.

The urine collection container will preferably hold a volume of e.g. up to 500 ml of urine. The urine collection container may also act as a portal for drug administration.

A Luer fitting may be included at the top of the urine collection container so that the container and the catheter can be irrigated if necessary to clear any accumulated debris. The system optionally includes provision for irrigating the system. For example, normal saline can be flushed through the system.

The second aspect of the invention also provides a kit of parts for assembling a urine collection system, the kit comprising:

a suprapubic urethral catheter comprising a hollow tube having sequentially, a suprapubic section, a bladder section and a urethral section, wherein, the bladder section comprises one or more drainage holes in the wall of the tube;
an abdominal stop attachable to the suprapubic section; an abdominal urine collection pouch connected to the suprapubic section, providing a flowpath from the bladder section to the container, and thus providing a low pressure reservoir; and
optionally a non-return cuff attachable to the bladder section between the one or more drainage holes and the abdominal stop, wherein the non-return cuff has a projecting portion projecting outwardly from the tube which is deformable towards the tube and which resists deformation away from the tube; and
optionally a waistband.

The first and second aspects of the invention may be combined e.g. to provide an Alternative Catheter System (ACS), as named by the Applicant. One embodiment of such an ACS is shown in FIGS. 5a and 5b.

The invention includes any combination of the aspects and preferred features described herein except where such a combination is clearly impermissible or expressly avoided.

### BRIEF DESCRIPTION THE DRAWINGS

Embodiments of the present invention will now be described, by way of example, with reference to the accompanying diagrammatic drawings, in which:

FIG. 1 shows a part cut away view of an embodiment of a catheter of the present invention.

FIG. 2 shows a part cut away view of an embodiment of a catheter of the present invention.

FIG. 3 shows an abdominal stop according to an embodiment of the present invention.

FIGS. 4a to 4e show a non-return cuff according to an embodiment of the present invention. FIG. 4a is a 3-D view; FIG. 4b is a longitudinal cross-sectional view;

FIG. 4c is an enlarged view of the aspect “A” circled in FIG. 4b; FIG. 4d is a side view; and FIG. 4e is an end view.

FIGS. 5a and 5b show one embodiment of the invention in which the first and second aspects of the invention are combined. The embodiment shown consists of a catheter with a non-return cuff and the catheter is attached to an abdominal collection pouch. The Applicant has named this embodiment the Alternative Catheter System (ACS). FIG. 5a shows a sagittal view of this embodiment in-situ in a male patient. FIG. 5b shows a frontal view of this embodiment in-situ in a male patient.

### DETAILED DESCRIPTION

The following detailed description of some embodiments of the invention should be read with reference to the drawings, wherein like reference numerals indicate like elements throughout the several views.

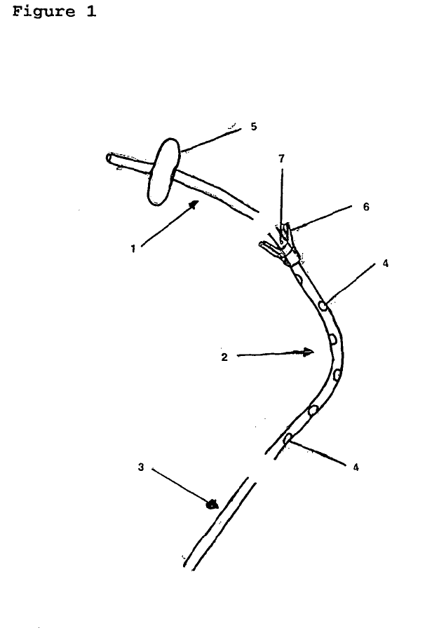

Referring to FIG. 1, there is illustrated a part cut away view of a suprapubic urethral catheter according to an embodiment of the present invention.

In this embodiment, the catheter includes a flexible and hollow tube which may be described in three sections, namely a suprapubic section 1, a bladder section 2 and a urethral section 3. The tubing may be continuous, or there may be two or three sections of tubing joined together.

The suprapubic section 1 passes through an abdominal stop 5 (also referred to as an abdominal flange) which can be adjusted by sliding it along the catheter before the catheter enters the abdominal wall. The upper end of this section can be cut to a suitable length for the patient and can be fitted to an abdominal urine collection pouch or a drainage funnel or connector (not shown).

The bladder section 2 comes in various lengths depending on the size of the bladder. At its suprapubic or upper end it is fitted with a non-return cuff 6 that prevents the catheter from extruding retrogradely from the bladder and at its lower end, a radio-opaque, ultrasound or other marker (not shown in FIG. 1) denotes its junction with the urethral section. A plurality of drainage eyeholes 4 are placed along the whole length of this section which enables the bladder to be drained completely of urine towards the urethral or lower end assisted by gravity or if this is closed through the upper end into a urine collection pouch (not shown in FIG. 1).

The urethral section 3 is a tube of thinner wall thickness which can be cut to length appropriate to the individual patient and fitted into a drainage funnel or connector (not shown in FIG. 1). A standard urethral valve (not shown in FIG. 1) which can be opened or closed can be inserted into the drainage funnel to store in or empty urine from the bladder. A urine collection bag can be fitted to the catheter valve at the urethral end if necessary.

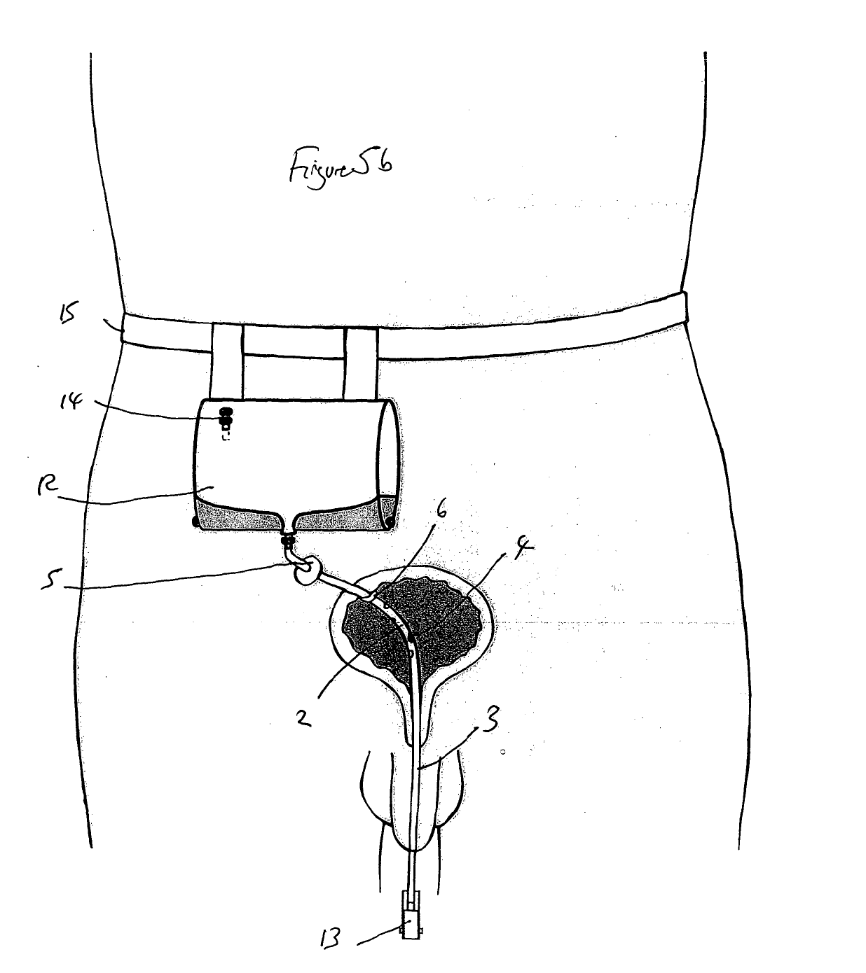

When located within a patient, the suprapubic end of the suprapubic section 1 of the catheter first passes through the abdominal stop 5, which grips and retains the catheter in position on the abdominal wall of the user, and then through the abdominal wall along a suprapubic track to join the bladder section 2 which is positioned in the bladder to drain urine. The bladder section leads on to the urethral section 3 which is positioned in the urethra. The location of the catheter within a patient can be seen from FIGS. 5a and 5b.

Referring to FIG. 1 again, the bladder section 2 with the plurality of drainage eyeholes 4 includes a non-return cuff 6 at its upper end which prevents the catheter from extruding retrogradely out of the suprapubic track in the abdominal wall. Small lateral irrigation eyeholes 7 are placed in the catheter tubing of the bladder section positioned within the projecting portion of the cuff to wash out any accumulated debris.

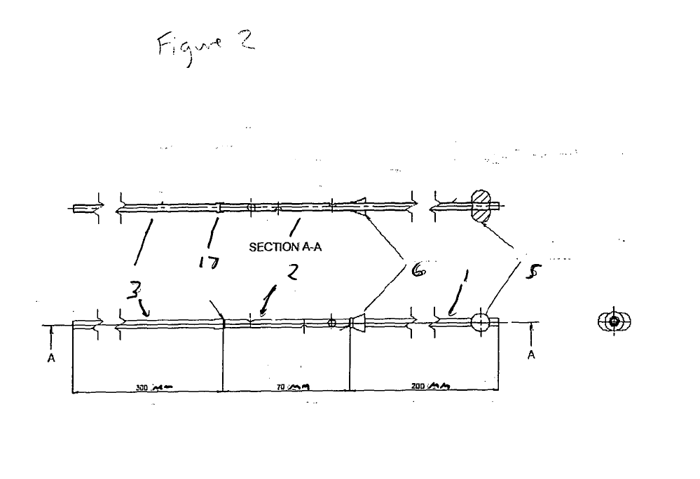

Referring to FIG. 2, there is illustrated one embodiment of a catheter according to the present invention. A non-return cuff 6 of a frusto-conical shape, and an abdominal stop 5 are shown attached to the catheter. Also shown is a radio-opaque or ultrasound marker 17 at the junction between the bladder section 2 and the urethral section 3.

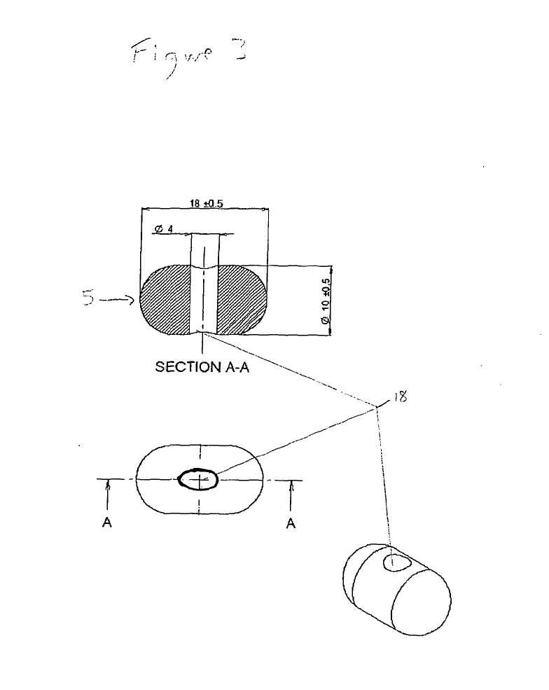

Referring to FIG. 3, there is illustrated one embodiment of an abdominal stop 5 according to the present invention. As can be seen, there is an oval channel 18 through the abdominal stop for receiving and gripping the suprapubic section of the catheter.

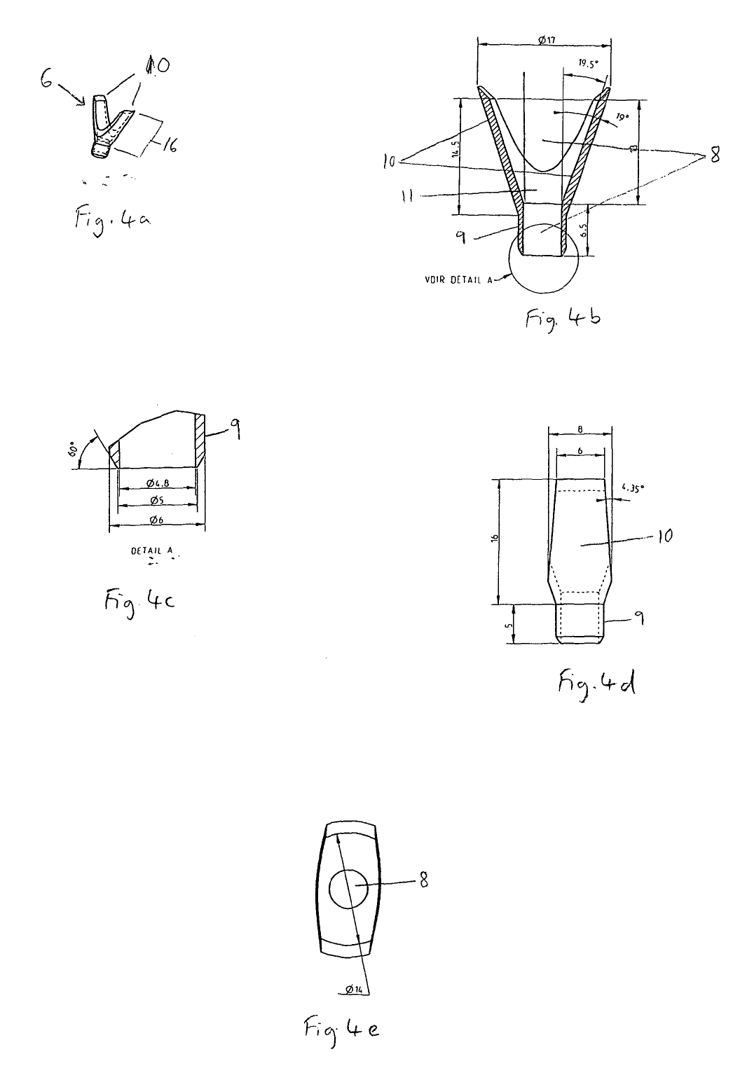

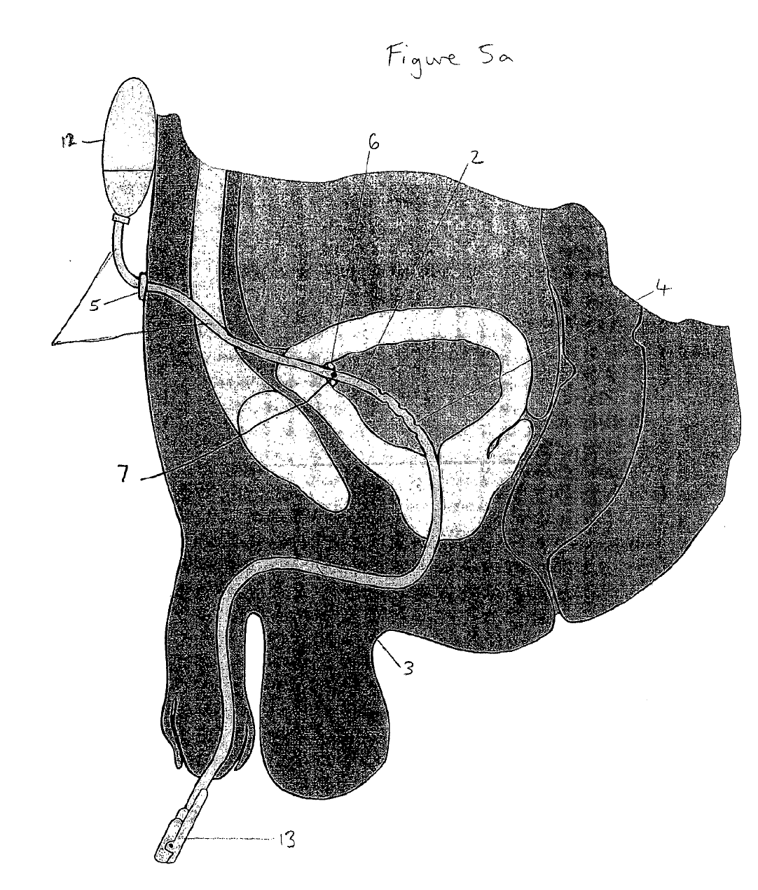

Referring to FIGS. 4a to 4e there is illustrated a non-return cuff 6 according to one embodiment of the present invention. FIG. 4a shows a non-return cuff having a projection portion 16 comprising two inclined wings 10. In other words the projection portion is substantially Vshaped. FIG. 4b shows a longitudinal cross-section of the same embodiment of the non-return cuff. As can be seen from FIG. 4b, the cuff has a channel 8 for receiving a catheter. The channel 8 extends throughout the centre of the cuff and between the two inclined wings 10. In other words the two inclined wings 10 are positioned on opposing sides of the channel 8. The inclined wings are joined by a resiliently deformable element 11 which biases the wings apart from each other. As can be seen from FIG. 4b, at the end of the wings, where the distance between the wings is smallest, a sleeve 9 is formed around part of the channel 8. The ends of the sleeve 9, furthest from the wings, taper towards the channel. This is for ease of insertion of the cuff into a bladder along a suprapubic track defined by a catheter held within the channel of the cuff. This tapering can be most clearly seen from FIG. 4c which provides an enlarged view of the area “A” circled in FIG. 4b.

The structural configuration of the cuff is designed to balance the need for flexibility to allow ease of insertion with strength and stiffness to enable its non-return function. In the embodiment shown in FIG. 4d, the wings 10 have a distorted elongated polygonal shape, in which the widest part of the elongated polygon is near the end of the wings being closest to the sleeve, as shown in FIG. 4d. This shape provides the arms with strength to resist deformation away from the channel whilst at the same time minimising any enlargement of the suprapubic track and the aperture into the bladder defined by the catheter on insertion.

Referring to FIGS. 5a and 5b, there is illustrated a urine collection system according to an embodiment of the invention named the “Alternative catheter system (ACS)” by the Applicant. In FIGS. 5a and 5b, an embodiment of the second aspect of the invention is shown combined with an embodiment of the first aspect of the invention. The ACS is shown in situ in a male patient in FIGS. 5a and 5b.

Regarding the first aspect of the invention, FIG. 5a shows a catheter having an abdominal stop 5 attached to the suprapubic section 1 and positioned against the abdomen, drainage holes 4 in the bladder section and a non-return cuff 6 attached to the bladder section 2 upstream of the drainage holes 4 (i.e. between the drainage holes and the abdominal stop) and inserted into the bladder.

Regarding the second aspect of the invention, FIGS. 5a and 5b show the upper or suprapubic end of the catheter, beyond the abdominal stop 5, attached to an abdominal urine collection pouch 12 which will hold a volume typically up to 500 ml of urine. There is no valve between the catheter and the pouch. Urine can flow freely from the bladder into the pouch as the bladder fills or if the bladder muscle contracts. When the bladder muscle relaxes, urine can return into the bladder. This system allows the bladder to fill normally and if the bladder pressure rises to a threshold pressure, for example as a result of a contraction of the bladder (detrusor) muscle, urine enters the abdominal urine collection pouch and then returns to the bladder once the pressure falls below the threshold, for example as the bladder muscle relaxes. Thus a low pressure urinary reservoir is maintained.

The bladder and any urine collection pouch 12 are emptied by releasing the catheter valve 13 at the lower end of the catheter.

The catheter and the pouch provide a closed urine collection system. A Luer or similar fitting 14 may be included at the top of the pouch (e.g. as shown in FIG. 5b) so that the pouch and catheter can be irrigated if necessary to clear any accumulated debris.

To flush the system, normal saline or other standard bladder washout solutions can be used to irrigate the bladder either through the Luer lock on the abdominal urine collection pouch or through the drainage funnel (not shown) at the upper end of the catheter. By releasing the catheter valve at the lower end of the catheter, the bladder can be flushed on a regular basis, the frequency depending on the amount of debris accumulating in the urine.

In one embodiment, as can be seen from the FIG. 5b, the abdominal urine collection pouch 12 is attached to the user's abdomen by a waistband 15.

The manner of introduction of the catheter of the present invention will now be described. A transurethral flexible cystoscopy should initially be performed to exclude the presence of pathology in the urethra or bladder such as stricture, tumour or stones. The urethral end of the ACS catheter is inserted through the suprapubic cystotomy and when this becomes visible in the bladder, the end is held in a pair of endoscopic grabbing forceps. The cystoscope, grabbing forceps and catheter are withdrawn from the urethra.

Gentle traction is applied to the urethral end of the catheter until the non-return cuff on the bladder section of the catheter has been passed through the abdominal wall and entered the bladder. Gentle traction is then applied to the upper or suprapubic end until the non-return cuff engages with the bladder wall thus preventing further withdrawal of the catheter from the bladder. Having reached that point, the abdominal flange is adjusted by sliding it along the catheter until it rests gently against the abdominal wall. In that way the catheter is held in place between the flange on the surface of the body and the non-return cuff within the bladder.

With the catheter in situ, the suprapubic section of the catheter is trimmed and attached to the abdominal urine collection pouch following the instructions provided with the pouch. After trimming the urethral section of the catheter to a suitable length for the patient, a drainage funnel is fitted to the lower end of the catheter and a catheter valve introduced into this.

To change the catheter, one possibility is to detach the abdominal urine collection pouch from the suprapubic section of the catheter above the abdominal flange. The drainage funnel is detached from the urethral end of the catheter. A guidewire is passed through the catheter from the suprapubic end until it emerges from the end of the urethral section. Whilst the guidewire is held securely, the abdominal flange is removed from the suprapubic section by sliding it along to the upper end of the catheter following which the used catheter is removed from the urethral end. The new ACS is passed over the guide wire from the suprapubic end until it emerges from the urethra, the guide wire is removed and the same procedure as for the initial catheterisation is followed, securing the non-return valve in the bladder and adjusting the abdominal flange before attaching the abdominal urine collection pouch at the suprapubic end and drainage funnel at the urethral end. A standard catheter valve is introduced into the drainage funnel.

Alternatively, the abdominal urine collection pouch may be detached from the suprapubic section of the catheter above the abdominal flange. Holding the catheter firmly between the abdominal wall and the abdominal flange, the flange is removed by sliding it off the suprapubic end of the catheter and a small connecting rod is inserted into the lumen at the end of the suprapubic section. The lumen at the urethral end of the new catheter is then inserted over the connecting rod and when firmly attached, gentle traction is applied to the urethral end of the used catheter to pull it together with the new catheter through the abdominal wall, bladder and urethra until it emerges from the lower or urethral end. The used catheter and the connecting rod are removed from the new catheter and the same procedure is followed to introduce the non-return cuff into the bladder, adjust the abdominal flange and attach the abdominal urine collection pouch and drainage funnel into the suprapubic and urethral ends of the new catheter respectively. A standard catheter valve is introduced into the drainage funnel.

The bladder and the urine collection pouch are emptied by releasing the catheter valve 13 at the lower end of the ACS. To flush the urine collection pouch and bladder, irrigation can be performed through the Luer or similar fitting 14 on the abdominal urine collection pouch using any standard bladder instillation or washout solution and by releasing the catheter valve 13.

The ACS provides a versatile means of draining the bladder. As an alternative to the abdominal urine collection pouch, a drainage funnel can be attached to the upper or suprapubic section of the catheter. With a drainage funnel at both ends, the bladder can be drained or flushed with a standard bladder irrigation fluid from either or both ends or using a catheter valve or a urine collection bag at one end and a spigot at the other. Bladder irrigation using standard bladder washout and other solutions can be performed from either end.

### REFERENCES

1. Saint S, Elmore J G, Sullivan S D, Emerson S S, Koepsell T D. The efficacy of silver alloy-coated urinary catheters in preventing urinary tract infection: a meta-analysis. Am J Med 1998: 105:236-241).
2. Maki D G, Tambyah P A. Engineering Out the Risk for infection with Urinary Catheters. Emerging Infectious Diseases 2001: 7(2):342-347.
3. Saint S. Clinical and economic consequences of nosocomial catheter-related bacteriuria. Am J Infect Control 2000: 28(1):68-73.
4. Rosenthal V D. Device-associated nosocomial infections in limited-resources countries: findings of the International Nosocomial Infection Control Consortium (INICC). Am J Infect Control 2008: 36(10):S171-12.
5. Foley FEB. A Self-Retaining Bag Catheter. J Urol 1937: 38:140-143.
6. Warren, J W Catheter-associated urinary tract infections Infect Dis North Am 11 (3): 609-622 1997.
7. Kohler-Ockmore J. and Feneley R C L. Long-term catheterisation of the bladder: prevalence and morbidity. British Journal of Urology. 77: 347-351 1996
8. Hellstrom L, Ekeland P, Milsom I, and Mellstrom D. Prevalence of incontinence: use of aids. Age and Ageing. 19: 383-389 1990.
9. Kunin, C M. Can we build a better urinary catheter? N Eng J Med 319 (6):365-366, 1988.

1. A suprapubic urethral catheter comprising:
a hollow tube having sequentially a suprapubic section, a bladder section and a urethral section;
wherein, there is an abdominal stop attached to the suprapubic section; and
wherein, the bladder section comprises one or more drainage holes in the wall of the tube and there is a non-return cuff attached to the bladder section between the one or more drainage holes and the abdominal stop; and
wherein, the non-return cuff has a projecting portion projecting outwardly from the tube which is deformable towards the tube and which resists deformation away from the tube.

2. A suprapubic urethral catheter according to claim 1, wherein the projecting portion of the cuff comprises a wing inclined away from the tube, wherein the end of the wing furthest from the tube is nearest the abdominal stop.

3. A suprapubic urethral catheter according to claim 2, wherein the projecting portion of the cuff comprises two inclined wings.

4. A suprapubic urethral catheter according to claim 3, wherein, the non-return cuff includes a channel for receiving the tube of the catheter, and the two inclined wings are positioned on opposite sides of the channel.

5. A suprapubic urethral catheter according to claim 4, wherein the two inclined wings are joined by a resiliently deformable element and the wings are biased apart from each other by the resiliently deformable element.

6. A suprapubic urethral catheter according to claim 5, wherein at the end of the wing furthest from the abdominal stop, the non-return cuff has a sleeve formed around the channel and attached to the tube.

7. A suprapubic urethral catheter according to claim 6, wherein at the end of the sleeve furthest from the abdominal stop, the walls of the sleeve taper towards the tube.

8. A suprapubic urethral catheter according to claim 3, wherein each wing is shaped as a distorted elongated polygon, wherein the polygon is elongated along the length of the wing, and the widest part of the hexagon is located towards the end of the wings being furthest from the abdominal stop.

9. A suprapubic urethral catheter according to claim 1, wherein there is a plurality of said drainage holes located along the bladder section.

10. A suprapubic urethral catheter according to claim 1, wherein there is at least one irrigation hole in the wall of the tube positioned in the bladder section within the projecting portion of the non-return cuff.

11. A suprapubic urethral catheter according to claim 1, wherein the abdominal stop has a channel for receiving and gripping the suprapubic section of the catheter.

12. A suprapubic urethral catheter according to claim 11, wherein the position of the abdominal stop along the suprapubic section is adjustable.

13. A suprapubic urethral catheter according to claim 1, wherein there is a radio-opaque or ultrasonic marker at the junction between the bladder section and the urethral section.

14. A suprapubic urethral catheter according to claim 1, wherein the suprapubic section is connected to a drainage funnel or connector.

15. A suprapubic urethral catheter according to claim 1, wherein the urethral section is connected to a drainage funnel or connector.

16. A suprapubic urethral catheter according to claim 15, wherein the drainage funnel or connector includes a valve for opening or closing the flowpath through the urethral section.

17. A kit of parts for assembling a self-retaining suprapubic urethral catheter, the kit comprising:
a suprapubic urethral catheter comprising a hollow tube having sequentially a suprapubic section, a bladder section and a urethral section, wherein the bladder section comprises one or more drainage holes in the wall of the tube;
an abdominal stop attachable to the suprapubic section;
a non-return cuff attachable to the bladder section between the one or more drainage holes and the abdominal stop, wherein the non-return cuff has a projecting portion projecting outwardly from the tube which is deformable towards the tube and which resists deformation away from the tube.

18. A urine collection system comprising:
a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, providing a flowpath from the bladder section to the container, and thus providing a low pressure reservoir.

19. A urine collection system according to claim 18, including a waistband for attaching the urine collection container to a patient.

20. A urine collection system according to claim 18, wherein the urine collection container is a pouch.

21. A urine collection system according to claim 18, wherein the urine collection container includes a Luer fitting.

22. A kit of parts for assembling a urine collection system, the kit comprising:
a suprapubic urethral catheter comprising a hollow tube having sequentially, a suprapubic section, a bladder section and a urethral section, wherein the bladder section comprises one or more drainage holes in the wall of the tube; and
an abdominal stop attachable to the suprapubic section;
an abdominal urine collection pouch connected to the suprapubic section, providing a flowpath from the bladder section to the pouch, and thus providing a low pressure reservoir.

23. A kit of parts according to claim 22, the kit further comprising:
a non-return cuff which is attachable to the bladder section between the one or more drainage holes and the abdominal stop, wherein the non-return cuff has a projecting portion projecting outwardly from the tube which is deformable towards the tube and which resists deformation away from the tube.

24. A kit of parts according to claim 22, the kit further comprising a waistband.

25. A urine collection system according to claim 18, wherein the suprapubic urethral catheter is one comprising:
a hollow tube having sequentially a suprapubic section, a bladder section and a urethral section;
wherein, there is an abdominal stop attached to the suprapubic section; and
wherein, the bladder section comprises one or more drainage holes in the wall of the tube and there is a non-return cuff attached to the bladder section between the one or more drainage holes and the abdominal stop; and
wherein, the non-return cuff has a projecting portion projecting outwardly from the tube which is deformable towards the tube and which resists deformation away from the tube.
